(19) United States
(12) Patent Application Publication
(10) Pub. No.: US 2025/0235390 A1
(43) Pub. Date: Jul. 24, 2025

(54) Composition and Process for Treating a Disorder in the Oral Cavity of a Mammal

(71) Applicant: Solventum Intellectual Properties Company, Maplewood, MN (US)

(72) Inventors: Henning Hoffmann, Windach (DE); Rüediger Hampe, Puergen (DE); Peter Osswald, Tuerkheim (DE); Joachim Zech, Kaufering (DE); Viola Baumgärtel, Muenchen (DE); Marc Peuker, Schondorf am Ammersee (DE)

(21) Appl. No.: 18/699,853

(22) PCT Filed: Oct. 14, 2022
(86) PCT No.: PCT/IB2022/059877

§ 371 Date: Apr. 9, 2024

(30) Foreign Application Priority Data
Oct. 20, 2021 (EP) 21203585.1

(51) Int. Cl. A61K 6/20; A61K 6/70; A61K 6/898

(52) U.S. Cl. CPC A61K 6/20; A61K 6/70; A61K 6/898

(57) ABSTRACT

The invention relates to a composition, in particular to a swellable composition, for use in a process of treating a disorder in the oral cavity of a mammal. The composition comprises a guanidinyl-containing polymer, carrageenan, filler and a paste-forming component. During the process an active substance for treating the disorder is applied to and absorbed by the composition. The composition can be used for various purposes, including treating calculus, treating periodontitis, treating tissue lesions and for endodontic treatments.

### FIELD OF INVENTION

The invention relates to a composition, in particular to a swellable composition, for use in a process of treating a disorder in the oral cavity of a mammal.

The composition comprises a guanidinyl-containing polymer, carrageenan, filler and a paste-forming component. During the process an active substance for treating the disorder is applied to and absorbed by the composition.

The composition can be used for various purposes, including treating calculus, treating periodontitis, treating tissue lesions and for endodontic treatments.

### BACKGROUND

It is sometimes difficult to apply an active substance to a selected surface area in the oral cavity and fix the active substance at the desired position for a longer period of time due the continuous flow of saliva, tongue movement, rinsing procedures or the consumption of food and drinks.

The application of an active substance may become relevant for example in the area of calculus removal, treating periodontitis, endodontic treatments and related or similar procedures.

With respect to calculus removal various procedures are described in the art:

US 2020/0315942 A1 (Gong et al.) relates to methods and kids for removing calculus from a tooth. The method can include applying a component A comprising a hydrogen peroxide or a precursor thereto and a component B the comprising a catalase to the tooth, thereby generating oxygen; and removing at least a part of the calculus from the tooth.

WO 2018/075150 A1 (3M) describes a method of removing calculus from a tooth comprising: applying a component A to the tooth, wherein component A comprises a metal ion selected from the group of lithium ion, magnesium ion, calcium ion, a precursor thereto, and a combination thereof; and an aprotic base having a pK_{b} in water of greater than 15.4, or a precursor thereto; applying a component B to the tooth, wherein component B comprises a bicarbonate ion or a precursor thereto; wherein components A and B are applied simultaneously or sequentially to the tooth, thereby generating a gas to soften and/or loosen at least part of the calculus on the tooth; and removing at least a part of the calculus from the tooth.

WO 2018/128704 A2 (3M) describes a medical composition comprising guanidinyl-containing polymer(s) and polyanionic polymer(s). The medical composition is useful for absorbing water-containing fluids and can be used as dental retraction composition or as part of a medical treatment device.

WO 2017/223161 A1 (3M) describes methods and kids for removing calculus from a tooth, wherein the method includes applying a component A comprising a hydrogen peroxide or precursor thereto and a component B comprising catalase to the tooth, thereby generating oxygen and removing at least a part of the calculus from the tooth.

However, in particular low viscous compositions may tend to drip from the treated surface or being washed off quickly by saliva before an active substance in the composition is able to interact and/or weaken the calculus for a sufficiently long time period.

### SUMMARY OF INVENTION

Thus, there is a need for a composition being able to adhere to moist surfaces, especially in the oral cavity.

The composition should be able to act as carrier for agents or active substances and should also be able to absorb these or other components on demand.

If the composition is used in the oral cavity, the components of the composition should further be non-toxic and physiologically acceptable.

The composition should in particular be useful for treating various disorders in the oral cavity, including calculus.

One or more of these objectives can be achieved by the compositions and processes described in the present text and claims.

In one embodiment the present invention features a composition A for use in a process of treating a disorder in the oral cavity of a mammal, composition A comprising a guanidinyl-containing polymer, carrageenan, a filler, a paste-forming component, optionally additives, the process comprising the steps of applying composition A to a selected surface area in the oral cavity of the mammal, bringing a composition B into contact with composition A, composition B comprising agent X, letting composition A absorb at least a portion of composition B, leaving composition A on the surface onto which composition A has been applied for at least 10 s, removing composition A from the selected surface area.

Agent X is typically a chemical substance being able to chemically react or interact with soft or hard dental tissue in the oral cavity.

The invention also relates to a kit of parts comprising Part A and Part B and optionally an instruction for use, Part A comprising composition A, Part B comprising composition B, composition A and composition B being as described in the claims and description.

A further embodiment of the invention is directed to a process of treating calculus in the oral cavity of a mammal, the process comprising the steps of a) applying a composition A1 containing a guanidinyl-containing polymer, carrageenan, a paste-forming component and filler to a tooth having calculus on its surface, b) bringing a composition B1 comprising hydrogen peroxide or a hydrogen peroxide precursor into contact with composition A1, c) bringing a composition C1 comprising a hydrogen peroxide decomposition component into contact with composition A1, d) leaving composition A1 on the treated surface for at least 10 s, in particular for a time sufficient for the oxygen generated by the hydrogen peroxide decomposition component from the composition comprising hydrogen peroxide or a hydrogen peroxide precursor to migrate into the calculus, e) removing composition A1 from the treated surface, f) optionally removing the calculus from the tooth, wherein steps b) and c) can also be carried out in reversed order, and wherein the guanidinyl-containing polymer, carrageenan, the paste-forming component, the filler, the hydrogen peroxide or hydrogen peroxide precursor, and the hydrogen peroxide decomposition component are those as described in the claims and description.

Described is also a process of producing the composition as described in the claims and description.

Unless defined differently, for this description the following terms shall have the given meaning:

The term “compound” or “component” is a chemical substance which has a certain molecular identity or is made of a mixture of such substances, e.g., polymeric substances.

A “water or liquid absorbing composition” is a composition being able to absorb water or liquid in an amount of at least 50 wt. % or at least 100 wt. % or at least 200 wt. % with respect to the weight of the composition.

A “liquid” is any solvent being able to at least partially disperse, dissolve or suspend the components being present in the composition at ambient conditions (e.g. 23° C.).

A “paste” is a material that typically consist of a suspension of granular material in a liquid. Pastes can be classified by their viscosity or their consistency comparable to dental impression material (cf. ISO 4823).

A “particle” means a substance being a solid having a shape which can be geometrically determined. The shape can be regular or irregular. Particles can typically be analysed with respect to e.g. particle size and particle size distribution.

The particle size (d50) of a powder can be obtained from the cumulative curve of the grain size distribution. Respective measurements can be done using commercially available granulometers (e.g. Malvern Mastersizer 2000). “D” represents the diameter of powder particles and “50” refers to the volume percentage of the particles. Sometimes, the 50% is also expressed as “0.5”. For example, “(d50)=1 μm” means that 50% of the particles have a size of 1 μm or less.

The terms “crosslinking”, “hardening”, “setting”, “curing” or “curable” are used interchangeable, all referring to the formation of material with a higher molecular weight and/or to the formation of a material having a higher viscosity, by creating a network due to chemical and/or physical interaction.

A “hardening-”, “curing-” or “setting-reaction” is a reaction, wherein physical properties such as viscosity, and tensile strength of a composition change over the time due to a chemical or physical reaction between the individual components.

“Radiation curable” means that the component (or composition, as the case may be) can be cured by applying radiation, preferably electromagnetic radiation with a wavelength in the visible light spectrum (380 to 740 nm) under ambient conditions and within a reasonable time frame (e.g. within about 60, 30 or 10 s).

“Phyllosilicates” are silicates: forming sheets of silicate tetrahedra with Si₂O₅. Phyllosilicates can be further divided in sub-groups, e.g. according to the number of sheets or layers arranged with each other.

Within the meaning of the present text, phyllosilicates are divided in the following subgroups: silicate minerals of the 2:1 layer type group and silicate minerals of the 1:1 layer type group.

Clay minerals belong to the group of phyllosilicates can be characterized by the number of layers linked or arranged with each other. This classification is also used in the present text.

E.g., in kaolinite, having the ideal formula A1₂[Si₂O₅(OH)₄]), two single layers are linked or arranged with each other.

E.g. in muscovite, having the ideal formula KAL₂(AlSi₃O₁₀) (OH)₂ and belonging to the mica type group of minerals, three layers are linked or arranged with each other.

A “dental composition” is a composition which can or is to be used in the dental or orthodontic field.

A “tooth structure” is any tooth structure, prepared or ready for preparation by the dentist. It can be a single tooth or two or more teeth. A tooth structure is also referred to as hard dental tissue in contrast to soft dental tissue (e.g. gingiva).

“Dental calculus” (also referred to as dental tartar) means a mineralized dental biofilm filled with crystals of various calcium phosphates or dental plaque that has partially or completely calcified. It may be caused by the continual accumulation of minerals from fluids in the oral environment on plaque on the teeth. Dental calculus is a common oral condition afflicting humans and a variety of animal species and the presence of dental calculus may lead to periodontal diseases. The existing methods of removing dental calculus, which rely upon mechanical means such as scaling, are time consuming and laborious for dental professionals, and can be a painful and unpleasant experience for patients.

A “paste” is a material that typically consist of a suspension of granular material in a liquid. Pastes can be classified by their viscosity or their consistency comparable to dental impression material (cf. ISO 4823).

“Ambient conditions” mean the conditions which the composition described in the present text is usually subjected to during storage and handling. Ambient conditions may, for example, be a pressure of 900 to 1,100 mbar, a temperature of 10 to 40° C. and a relative humidity of 10 to 100%. In the laboratory ambient conditions are typically adjusted to 20 to 25° C. and 1,000 to 1,025 mbar (at maritime level).

As used herein, “a”, “an”, “the”, “at least one” and “one or more” are used interchangeably. Also herein, the recitations of numerical ranges by endpoints include all numbers subsumed within that range (e.g., 1 to 5 includes 1, 1.5, 2, 2.75, 3, 3.80, 4, 5, etc.).

Adding an “(s)” to a term means that the term should include the singular and plural form. E.g. the term “additive(s)” means one additive and more additives (e.g. 2, 3, 4, etc.).

Unless otherwise indicated, all numbers expressing quantities of ingredients, measurement of physical properties such as described below and so forth used in the specification and claims are to be understood as being modified in all instances by the term “about”.

The terms “comprise” or “contain” and variations thereof do not have a limiting meaning where these terms appear in the description and claims. “Consisting essentially of” means that specific further components can be present, namely those which do not materially affect the essential characteristic of the article or composition. “Consisting of” means that no further components should be present. The term “comprise” shall include also the terms “consist essentially of” and “consists of”.

A composition is “essentially or substantially free of” a certain component, if the composition does not contain said component as an essential feature. Thus, said component is not wilfully added to the composition either as such or in combination with other components or ingredient of other components. A composition being essentially free of a certain component usually does not contain that component at all. However, sometimes the presence of a small amount of the said component is not avoidable e.g. due to impurities contained in the raw materials used.

If not indicated otherwise, wt. % always refers to the weight of the whole composition mentioned.

### BRIEF DESCRIPTION OF FIGURES

FIGS. 1-5 exemplify the use of the composition described in the present text in a process of treating calculus.

### DETAILED DESCRIPTION

It has been found that the composition and processes described in the present text have a couple of advantageous properties.

The composition described in the present text contains a matrix acting as a carrier that can be loaded with (bio)active substances for different treatment purposes.

This matrix is formed essentially by the combination of a guanidinyl-containing polymer and carrageenan. Such a composition is able to absorb liquids and is able to form a hydrogel with water.

Further, due to its hydrophilic nature, the composition can adhere to tissue in the oral cavity.

Upon placement of the composition on a selected surface area in the oral cavity, the active substance having been absorbed by the composition during the treatment process can then interact with the oral environment and the active substance can be released or delivered to the desired region.

However, the composition is also easily removable from the tissue by equipment being available to a dental practitioner, e.g. water-spray, if desired.

Using a hardenable composition may provide additional benefits.

In this respect due to the possibility to cure the top or outer layer of the composition, the remaining (non-hardened) composition below the cured top layer can stay in place on the selected surface area in the oral cavity for a longer time period even if liquids or the tongue touches it and thus can contribute to a longer and more effectful treatment of the situation to be treated.

The invention relates to a composition A for use in a process of treating a disorder in the oral cavity of a mammal by applying an agent X to a selected surface area in the oral cavity of the mammal.

Composition A comprises one or more guanidinyl-containing polymers.

The term “guanidinyl-containing polymer” includes also polymers where the guanidinyl moiety is present in its protonated form including the salts thereof (in particular chloride and sulphate salts).

Suitable polymers include polyvinylamine, poly(N-methylvinylamine), polyallylamine, polyallylmethylamine, polydiallylamine, poly(4-aminomethylstyrene), poly(4-aminostyrene), poly(acrylamide-co-methylaminopropylacrylamide), poly(acrylamide-co-aminoethylmethacrylate), polyethylenimine, polypropylenimine, polylysine, polyaminoamides, polydimethylamine-epichlorohydrin-ethylenediamine, polyaminosiloxanes, dendrimers formed from polyamidoamine and polypropylenimine, biopolymers, polyacrylamide homo- or copolymers, amino-containing polyacrylate homo- or copolymers.

For some embodiments, the preferred amino-containing polymers include polyaminoamides, polyethyleneimine, polyvinylamine, polyallylamine, polydiallylamine and acrylamide-based polymers.

As used herein, the term “guanidinyl” refers to a group of the following formula

—NR³—C(═NR⁴)—NR⁴R⁵

If the guanidinyl group is part of a pendant group, the group R³ refers to hydrogen, C1-C12 (hetero)alkyl, or C5-C12 (hetero)aryl.

If the guanidinyl group is part of the backbone of the polymer, the group R³ can refer to a residue of a polymer chain.

Each group R⁴ is independently hydrogen, C₁-C₁₂ (hetero)alkyl, or C₅-C₁₂ (hetero)aryl. Group R⁵ is hydrogen, C₁-C₁₂ (hetero)alkyl, C₅-C₁₂ (hetero)aryl, or a group of formula —N(R⁴)₂.

The guanidinyl group can be part of a biguanidinyl group that is of formula —NR³—C(═NR⁴)—NR⁴—C(═NR⁴)—NR⁴R⁵ where the groups R³, R⁴, and R⁵ are the same as defined above.

Although any guanidinyl-containing polymer can be used in the cationic form, this polymer is often of Formula (I).

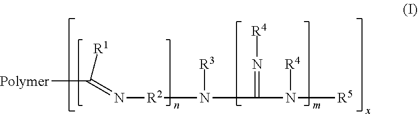

In Formula (I), the group R¹ is hydrogen, C₁-C₁₂ (hetero)alkyl, or C₅-C₁₂ (hetero)aryl, or a residue of the polymer chain. The group R² is a covalent bond, a C₂-C₁₂ (hetero)alkylene, or a C₅-C₁₂ (hetero)arylene. The group R³ is H, C₁-C₁₂ (hetero)alkyl, or C₅-C₁₂ (hetero)aryl, or can be a residue of the polymer chain when n is 0. Each group R⁴ is independently hydrogen, C₁-C₁₂ (hetero)alkyl, or C₅-C₁₂ (hetero)aryl. The group R⁵ is hydrogen, C₁-C₁₂ (hetero)alkyl, C₅-C₁₂ (hetero)aryl, or—N(R⁴)₂. The variable n is equal to 0 or 1 depending on the precursor polymer used to form the guanidinyl-containing polymer. The variable m is equal to 1 or 2 depending on whether the cationic group is a guanidinyl or biguanidinyl group. The term “Polymer” in Formula (I) refers to all portions of the guanidinyl-containing polymer except the x groups of formula —[C(R¹)═N—R²-]ₙN(R³)—[C(═NR⁴)—NR⁴R⁵-]ₘ. The term x is a variable equal to at least 1 or in a range of 10 to 1,000 or 100 to 1,000.

Most guanidinyl-containing polymers have more than one guanidinyl group. The number of guanidinyl groups can be varied depending the method used to prepare the guanidinyl-containing polymer. For example, the number of guanidinyl groups can depend on the choice of precursor polymer selected for reacting with a suitable guanylating agent. In some embodiments, the variable x can be up to 1000, up to 500, up to 100, up to 80, up to 60, up to 40, up to 20, or up to 10.

The guanidinyl-containing polymer of Formula (I) is often the reaction product of (a) a precursor polymer and (b) a suitable guanylating agent.

The precursor polymer is often an amino-containing polymer or a carbonyl-containing polymer. When the precursor polymer is an amino-containing polymer, the variable n in Formula (I) is typically equal to 0. When the precursor polymer is a carbonyl-containing polymer, the variable n is equal to 1. If the guanylating agent contains a guanidinyl group or a precursor of a guanidinyl group, the variable m in Formula (I) is equal to 1. If the guanylating agent contains a biguanidinyl group or a precursor of a biguanidinyl group, the variable m in Formula (I) is equal to 2.

In embodiments where n is 0, the base polymer of the guanidinyl-containing polymer is often prepared by reaction of a suitable guanylating agent and an amino-containing polymer. In other embodiments, where n is 1, the guanidinyl-containing polymer is often prepared by reaction of a suitable guanylating agent and a carbonyl-containing polymer.

In those embodiments where n is 0 and the precursor polymer is an amino-containing polymer, the structure of the guanidinyl-containing polymer of Formula (I) can also be written more simply as the structure of Formula (II).

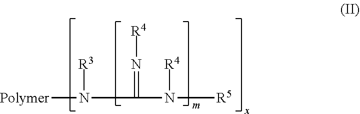

In Formula (II), the group R³ is hydrogen, C₁-C₁₂ (hetero)alkyl, or C₅-C₁₂ (hetero)aryl, or can be a residue of the polymer chain. When the guanidinyl group is part of a pendant group, R³ is hydrogen, C₁-C₁₂ (hetero)alkyl, or C₅-C₁₂ (hetero)aryl. Each R⁴ is independently hydrogen, C₁-C₁₂ (hetero)alkyl, or C₅-C₁₂ (hetero)aryl. The group R⁵ is hydrogen, C₁-C₁₂ (hetero)alkyl, or C₅-C₁₂ (hetero)aryl, or—N(R⁴)₂. The variable m is equal to 1 or 2. The term “Polymer” in the formula above refers to all portions of the guanidinyl-containing polymer except the x groups of formula —N(R³)—[C(═NR⁴)—NR⁴R⁵-]ₘ. The term x is a variable equal to at least 1 or in a range of 10 to 1,000 or 100 to 1,000.

The amino-containing polymer used as a precursor polymer to prepare a guanidinyl-containing polymer of Formula (II) can be represented by the formula Polymer-N(R³) H. As noted above, however, the amino-containing polymer typically has many groups-N(R³) H but Formula (I) shows only one for ease of discussion purposes only. The—N(R³) H groups can be a primary or secondary amino group and can be part of a pendant group or part of the backbone of the precursor polymer. The amino-containing polymers can be synthesized or can be naturally occurring biopolymers. Suitable amino-containing polymers can be prepared by chain growth or step growth polymerization procedures with amino-containing monomers. These monomers can also, if desired, be copolymerized with other monomers without an amino-containing group. Additionally, the amino-containing polymers can be obtained by grafting primary or secondary amine groups using an appropriate grafting technique.

The guanidinyl-containing polymer also includes polymers where the guanidinyl moiety is protonated including polymers having the following formula:

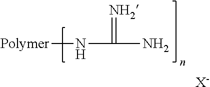

with X⁻ being selected from Cl⁻, Br⁻, I⁻, 1/2 SO₄²⁻, NO₃⁻, CH₃COO⁻, C₃H₇COO⁻ and n being a variable equal to at least 1 or in a range of 10 to 1,000 or 100 to 1,000.

Examples of amino-containing polymers suitable for use, which are prepared by chain growth polymerization include, but are not limited to, polyvinylamine, poly(N-methylvinylamine), polyallylamine, polyallylmethylamine, polydiallylamine, poly(4-aminomethylstyrene), poly(4-aminostyrene), poly(acrylamide-co-methylaminopropylacrylamide), and poly(acrylamide-co-aminoethylmethacrylate).

Examples of amino-containing polymers suitable for use, which are prepared by step growth polymerization include, but are not limited to, polyethylenimine, polypropylenimine, polylysine, polyaminoamides, polydimethylamine-epichlorohydrin-ethylenediamine, and any of a number of polyaminosiloxanes, which can be prepared from monomers such as aminopropyl-triethoxysilane, N-(2-aminoethyl)-3-aminopropyltrimethoxysilane, N-trimethoxysilylpropyl-N-methylamine, and bis(trimethoxysilylpropyl)amine.

Other useful amino-containing polymers that have primary or secondary amino end groups include, but are not limited to, dendrimers (hyperbranched polymers) formed from polyamidoamine (PAMAM) and polypropylenimine. Exemplary dendrimeric materials formed from PAMAM are commercially available under the trade designation “STARBURST (PAMAM) dendrimer” (e.g., Generation 0 with 4 primary amino groups, Generation 1 with 8 primary amino groups, Generation 2 with 16 primary amino groups, Generation 3 with 32 primary amino groups, and Generation 4 with 64 primary amino groups) from Aldrich Chemical (Milwaukee, WI). Dendrimeric materials formed from polypropylenimine are commercially available under the trade designation “DAB-Am” from Aldrich Chemical. For example, DAB-Am-4 is a generation 1 polypropylenimine tetraamine dendrimer with 4 primary amino groups, DAB-Am-8 is a generation 2 polypropylenimine octaamine dendrimer with 8 primary amino groups, DAB-Am-16 is a generation 3 polypropylenimine hexadecaamine with 16 primary amino groups, DAB-Am-32 is a generation 4 polypropylenimine dotriacontaamine dendrimer with 32 primary amino groups, and DAB-Am-64 is a generation 5 polypropylenimine tetrahexacontaamine dendrimer with 64 primary amino groups.

Examples of suitable amino-containing polymers that are biopolymers include chitosan as well as starch that is grafted with reagents such as methylaminoethylchloride.

Still other examples of amino-containing polymers include polyacrylamide homo- or copolymers and amino-containing polyacrylate homo- or copolymers prepared with a monomer composition containing an amino-containing monomer such as an aminoalkyl (meth)acrylate, (meth)acrylamidoalkylamine, and diallylamine.

For some embodiments, the preferred amino-containing polymers include polyaminoamides, polyethyleneimine, polyvinylamine, polyallylamine, and polydiallylamine.

Suitable commercially available amino-containing polymers include, but are not limited to, polyamidoamines that are available under the trade designations ANQUAMINE (e.g., ANQUAMINE 360, 401, 419, 456, and 701) from Air Products and Chemicals (Allentown, PA), polyethylenimine polymers that are available under the trade designation LUPASOL (e.g., LUPASOL FG, PR 8515, Waterfree, P, and PS) from BASF Corporation (Resselaer, NY), polyethylenimine polymers such as those available under the trade designation CORCAT P-600 from EIT Company (Lake Wylie, SC), and polyamide resins such as those available from Cognis Corporation (Cincinnati, OH) under the traded designation VERSAMID series of resins that are formed by reacting a dimerized unsaturated fatty acid with alkylene polyamines.

Guanidinyl-containing polymers can be prepared by reaction of the amino-containing polymer precursor with a guanylating agent.

Although all the amino groups of the amino-containing polymer can be reacted with the guanylating agent, there are often some unreacted amino groups from the amino-containing polymer precursor remaining in the guanidinyl-containing polymer. Typically, at least 0.1 mole percent, at least 0.5 mole percent, at least 1 mole percent, at least 2 mole percent, at least 10 mole percent, at least 20 mole percent, or at least 50 mole percent of the amino groups in the amino-containing polymer precursor are reacted with the guanylating agent. Up to 100 mole percent, up to 90 mole percent, up to 80 mole percent, or up to 60 mole percent of the amino groups can be reacted with the guanylating agent. For example, the guanylating agent can be used in amounts sufficient to functionalize 0.1 to 100 mole percent, 0.5 to 90 mole percent, 1 to 90 mole percent, 1 to 80 mole percent, 1 to 60 mole percent, 2 to 50 mole percent, 2 to 25 mole percent, or 2 to 10 mole percent of the amino groups in the amino-containing polymer.

Known guanylating agents for reaction with an amino-containing polymer precursor include, but are not limited to, cyanamide; O-alkylisourea salts such as O-methylisourea sulfate, O-methylisourea hydrogen sulfate, O-methylisourea acetate, O-ethylisourea hydrogen sulfate, and O-ethylisourea hydrochloride; chloroformamidine hydrochloride; 1-amidino-1,2,4-triazole hydro-chloride; 3,5-dimethylpyrazole-1-carboxamidine nitrate; pyrazole-1-carboxamidine hydrochloride; N-amidinopyrazole-1-carboxamidine hydrochloride; and carbodiimides such as dicyclohexyl-carbodiimide, N-ethyl-N′-(3-dimethylaminopropyl) carbodiimide, and diisopropylcarbodiimide. The amino-containing polymer may also be acylated with guanidino-functional carboxylic acids such as guanidinoacetic acid and 4-guanidinobutyric acid in the presence of activating agents such as EDC (N-[3-(dimethylamino) propyl]-3-ethylcarbodiimide hydrochloride), or EEDQ (2-ethoxy-1-ethoxycarbonyl-1,2-dihydroquinoline). Additionally, the guanidinyl-containing polymer may be prepared by alkylation with chloroacetone guanyl hydrazone, e.g. as described in U.S. Pat. No. 5,712,027 (Ali et al.).

Guanylating agents for the preparation of biguanide-containing polymers include sodium dicyanamide, dicyanodiamide and substituted cyanoguanidines such as N³-p-chlorophenyl-N¹-cyanoguanidine, N³-phenyl-N¹-cyanoguanidine, N³-alpha-naphthyl-N¹-cyanoguanidine, N³-methyl-N¹-cyanoguanidine, N³,N³-dimethyl-N¹-cyanoguanidine, N³-(2-hydroxyethyl)-N¹-cyanoguanidine, and N³-butyl-N¹-cyanoguanidine. Alkylene- and arylenebiscyanoguanidines may be utilized to prepare biguanide functional polymers by chain extension reactions. The preparation of cyanoguanidines and biscyanoguanidines is described in detail in Rose, F. L. and Swain, G. J. Chem Soc., 1956, pp. 4422-4425. Other useful guanylating reagents are described e.g. by Alan R. Katritzky et al., Comprehensive Organic Functional Group Transformation, Vol.6, p.640.

The guanidinyl-containing polymer formed by reaction of an amino-containing polymer precursor and a guanylating agent will have pendent or catenary guanidinyl groups of the Formula (III).

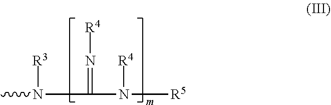

In Formula (III), the groups R³, R⁴, and R⁵ and the variable m are the same as defined above. The wavy line attached to the N(R³) group shows the position of attachment the group to the rest of the polymeric material. In most embodiments, the group of Formula (III) is in a pendant group of the guanidinyl-containing polymer.

In some embodiments, it may be advantageous to react the amino-containing polymer precursor to provide other ligands or groups in addition to the guanidinyl-containing group. For example, it may be useful to include a hydrophobic ligand, an ionic ligand, or a hydrogen bonding ligand. This can be particularly advantageous for the removal of certain microorganisms during the wiping of a microorganism-contaminated surface.

The additional ligands can be readily incorporated into the amino-containing polymers by alkylation or acylation procedures well known in the art. For example, amino groups of the amino-containing polymer can be reacted using halide, sulfonate, and sulfate displacement reactions or using epoxide ring opening reactions. Useful alkylating agents for these reactions include, for example, dimethylsulfate, butyl bromide, butyl chloride, benzyl bromide, dodecyl bromide, 2-chloroethanol, bromoacetic acid, 2-chloroethyltrimethylammonium chloride, styrene oxide, glycidyl hexadecyl ether, glycidyltrimethylammonium chloride, and glycidyl phenyl ether. Useful acylating agents include, for example, acid chlorides and anhydrides such as benzoyl chloride, acetic anhydride, succinic anhydride, and decanoyl chloride, and isocyanates such as trimethylsilylisocyanate, phenyl isocyanate, butyl isocyanate, and butyl isothiocyanate. In such embodiments 0.1 to 20 mole percent, preferably 2 to 10 mole percent, of the available amino groups of the amino-containing polymer may be alkylated and/or acylated.

The guanidinyl-containing polymer can be crosslinked. The amino-containing polymer can be crosslinked prior to reaction with the guanylating agent. Alternatively, the guanidinyl-containing polymer can be crosslinked by reaction of a crosslinker with remaining amino groups from the amino-containing polymer precursor or with some of the guanidinyl groups. Suitable crosslinkers include amine-reactive compounds such as bis- and polyaldehydes such as glutaraldehyde, bis- and polygylcidylethers such as butanedioldiglycidylether and ethyleneglycoldiglycidylether, polycarboxylic acids and their derivatives (e.g., acid chlorides), polyisocyanates, formaldehyde-based crosslinkers such as hydroxymethyl and alkoxymethyl functional crosslinkers, such as those derived from urea or melamine, and amine-reactive silanes, such as 3-glycidoxypropyltrimethoxysilane, 3-glycidoxypropyltriethoxysilane, 5,6-epoxyhexyltriethoxysilane, (p-chloromethyl)phenyltrimethoxysilane, chloromethyltriethoxysilane, 3-isocyanatopropyltriethoxysilane, and 3-thiocyanatopropyltriethoxysilane.

In other embodiments, the guanidinyl-containing polymer is of Formula (IV), which corresponds to Formula (I) where n is equal to 1.

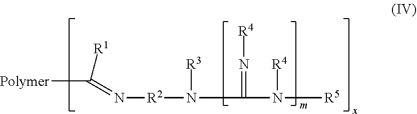

In Formula (IV), the group R¹ is hydrogen, C₁-C₁₂ (hetero)alkyl, or C₅-C₁₂ (hetero)aryl, or a residue of the polymer chain. If the guanidinyl-containing group is the reaction product of a guanylating agent and a carbonyl group that is part of the backbone of the polymer, R¹ is a residue of the polymer chain. Group R² is a covalent bond, a C₂-C₁₂ (hetero)alkylene, or a C₅-C₁₂ (hetero)arylene. Group R³ is hydrogen, C₁-C₁₂ (hetero)alkyl, or C₅-C₁₂ (hetero)aryl. Each R⁴ is independently H, C₁-C₁₂ (hetero)alkyl, or C₅-C₁₂ (hetero)aryl. Group R⁵ is hydrogen, C₁-C₁₂ (hetero)alkyl, or C₅-C₁₂ (hetero)aryl, or—N(R⁴)₂. The variable m is equal to 1 or 2. The term “Polymer” in Formula (I) refers to all portions of the guanidinyl-containing polymer except the x groups of formula —C(R¹)═N-R²—N(R³)—[C(═NR⁴)—NR⁴R⁵-]ₘ. The term x is a variable equal to at least 1 or in a range of 10 to 1,000 or 100 to 1,000.

Guanidinyl-containing polymers of Formula (IV) are the reaction product of a carbonyl-containing polymer and a suitable guanylating agent for reaction with a carbonyl group. The carbonyl-containing polymer used as a precursor polymer to prepare a guanidinyl-containing polymer of Formula (IV) can be represented by the formula Polymer-C(O)—R¹. The carbonyl-containing polymer precursor typically has many groups-C(O)—R¹ but Formula (IV) shows only one for ease of discussion purposes only. The carbonyl group-C(O)—R¹ is an aldehyde group (when R¹ is hydrogen) or a ketone group (when R¹ is a (hetero)alkyl or (hetero)aryl). Although the carbonyl-group can be part of the polymeric backbone or part of a pendant group from the polymeric backbone, it is typically in a pendant group.

If desired, the guanidinyl-containing polymers can be produced as described in US 2016/0115430 A1 (Swanson et al.), in particular in sections to [0080], the description of which is herewith incorporated by reference.

The guanidinyl-containing polymer is typically present in the following amounts: at least: 1 or 3 or 5 wt. %; utmost: 60 or 40 or 30 wt. %; range: 1 to 60 or 3 to 40 or 5 to 30 wt. %; wt. % with respect the weight of composition A.

Composition A described in the present text comprises one or more carrageenans.

Carrageenans or carrageenins are a family of sulphated polysaccharides that are typically extracted from red edible seaweeds.

There are three main varieties of carrageenan, which differ in their degree of sulfphation.

Kappa-carrageenan has one sulphate group per disaccharide. Iota-carrageenan has two sulphates per disaccharide. Lambda carrageenan has three sulphates per disaccharide. Other carrageenan(s) which are known are epsilon and μ.

With respect to the present text, the use of iota or lambda carrageenan(s) can sometimes be preferred.

Carrageenans are large, highly flexible molecules that curl forming helical structures. This gives them the ability to form a variety of different gels at room temperature.

Carrageenans are polysaccharides made up of repeating galactose units and 3,6 anhydrogalactose (3,6-AG), both sulphated and non-sulphated. The units are typically joined by alternating a-1,3 and β-1,4 glycosidic linkages.

If desired, the carrageenan(s) can be characterized by one or more of the following features: molecular weight (Mw; weight average): 10,000 to 1,000,000 g/mol or 20,000 to 500,000 g/mol; ester sulphate content: 25 to 40 wt. % or 25 to 30 wt. % with respect to the weight of the carrageenan.

A carrageenan having a molecular weight (Mw) in the range of 20,000 to 500,000 g/mol and ester sulphate content of 25 to 40 wt. % is sometimes preferred.

The carrageenan(s) is typically present in the following amounts: at least: 1 or 2 or 5 wt. %; utmost: 40 or 35 or 30 wt. %; range: 1 to 40 or 2 to 35 or 5 to 30 wt. %; wt. % with respect to the weight of composition A.

The ratio of guanidinyl-containing polymer(s) to carrageenan(s) is typically in a range of 4 to 1 to 1 to 4, or 3 to 1 to 1 to 3, or 2 to 1 to 1 to 2 with respect to weight.

According to one embodiment, the guanidinyl-containing polymer and the carrageenan component are used in essentially equal amounts with respect to weight.

A suitable composition may comprise the guanidinyl-containing polymer in an amount of to 30 wt. % and the carrageenan component in an amount of 10 to 30 wt. %.

Composition A described in the present text comprises one or more fillers.

A wide variety of inorganic, hydrophilic or hydrophobic fillers may be employed such as silicates, silicas (including quartz and cristobalite), alumina, magnesia, titania, inorganic salts, metallic oxides and glasses.

The sizes and surface areas of the filler particles can be adjusted to control the viscosity and thixotropicity of the resulting compositions.

Some or all of the fillers may be superficially treated with one or more silanating agents, as known to those of ordinary skill in the art. Such silanating may be accomplished through use of known halogenated silanes or alkoxysilanes or silazanes.

A combination of reinforcing and non-reinforcing fillers can also be used.

In this respect, the amount of reinforcing fillers can range from 1 to 10 wt. %, in particular from 2 to 5 wt. % with respect to the whole composition.

Typical reinforcing fillers include fumed silica, and the like. Pyrogenically-prepared highly-disperse silicic acids which have preferably been hydrophobized by surface treatment are sometimes preferred as reinforcing fillers. The surface treatment can be carried out, for example with dimethyldichlorosilane, hexamethyldisilazane, tetramethylcyclotetrasiloxane or polymethylsiloxane.

Typical non-reinforcing fillers are phyllosilicates, quartz, cristobalite, precipitated silicas, diatomaceous earth, alumina, magnesia, titanium dioxide, zirconium silicate, metallic oxides, barium sulphate, calcium carbonate, plaster, glass and the like.

The non-reinforcing fillers can be surface treated, e.g. silanated, or non-surface treated.

Typical average particle sizes are from 2 to 10 μm.

Filler(s) are typically present in the following amounts: at least: 3 or 5 or 10 wt. %; utmost: 70 or 60 or 50 wt. %; range: 3 to 70 or 5 to 60 or 10 to 50 wt. %; wt. % with respect to the weight of composition A.

The use of phyllosilicates as filler is sometimes preferred.

The nature and structure of the phyllosilicate(s) is not particularly limited unless the desired result cannot be achieved.

Phyllosilicates which can be used include layer type 1:1 silicate minerals, layer type 1:2 silicate minerals and mixtures of layer type 1:1 silicate minerals and layer type 1:2 silicate minerals.

Phyllosilicates from the layer type 1:1 silicate mineral which can be used include kaolinite, lizardite, halloysite and mixtures or combinations thereof, wherein kaolinite is sometimes preferred.

The particle size of the layer type 1:1 silicate mineral is not particularly limited, unless the resulting paste gets to inhomogeneous. The mean particle size is typically in a range between 0.01 and 100 μm or between 0.1 and 50 μm or between 1 and 25 μm.

The content of the layer type 1:1 silicate mineral in the composition is not particularly limited, unless the desired advantages cannot be obtained. If present, the layer type 1:1 silicate mineral is typically present in an amount of 3 wt. % to 55 wt. % or 5 wt. % to 50 wt. % with respect to the whole composition.

Phyllosilicates from the layer type 2:1 silicate minerals which can be used include mica minerals, talc-pyrophyllite minerals, smectite minerals, vermiculite minerals, illites minerals.

Specific examples include talc, willemseite, pyrophyllite, stevensite, saponite (from the talc-pyrophyllite type group of minerals), stevensite, sponite, sauconite, hectorite, montmorillonite, beidellite, nontronite, volkonskite (from the smectite type group of minerals), phlogopite, biotite, lepidolite, muscovite, illite, glauconite, celadonite (from the mica type group of minerals).

The particle size of the layer type 2:1 silicate mineral is not particularly limited, unless the resulting composition gets too inhomogeneous. The mean particle size is typically in the range of 0.01 to 100 μm or 0.1 to 50 μm or 1 to 25 μm.

The content of the layer type 2:1 silicate mineral in the composition is not particularly limited, unless the desired advantages cannot be obtained. If present, the layer type 2:1 silicate mineral is typically present in an amount of 3 wt. % to 55 wt. % or 5 wt. % to 50 wt. % with respect to the whole composition.

If a combination of layer type 1:1 silicate minerals and layer type 1:2 silicate minerals is used, the layer type 1:1 silicate mineral and the layer type 2:1 silicate mineral are typically present in the dental retraction composition in a certain weight ratio with respect to each other.

If a combination of phyllosilicate(s) is desired, the following mixtures can be used: layer type 1:1 silicates selected from kaolinite, lizardite, halloysite; layer type 1:2 silicates selected from mica minerals, talc-pyrophyllite minerals, smectite minerals, vermiculite minerals, illites minerals.

If present, the phyllosilicate(s) are typically present in the following amounts: at least: 3 or 5 or 8 wt. %; utmost: 60 or 55 or 50 wt. %; range: 3 to 60 or 5 to 55 or 8 to 50 wt. %; wt. % with respect to the weight of composition A.

Composition A also comprises a paste-forming component.

Paste-forming component(s) include those, which are able to form a paste with the other components present.

The paste-forming component can typically be characterized by one or more of the following features: a) being non-aqueous; b) being hydrophilic; c) having a boiling point above 100° C.; d) having a molecular weight (Mw) in the range of 60 to 10,000 g/mol, or 200 to 10,000 g/mol.

A combination of features a) and c), or a) and d); or a), c) and d) is sometimes preferred.

A molecular weight as outlined above can be beneficial because the risk of an undesired evaporation of the paste forming liquid(s) from the composition can be reduced.

The amount of paste-forming component in the composition is not particularly limited, unless the desired advantages cannot be obtained.

The paste-forming component(s) are typically present in the following amounts: at least: 1 or 5 or 10 wt. %; utmost: 60 or 55 or 50 wt. %; range: 1 to 60 or 5 to 55 or 10 to 50 wt. %; wt. % with respect to weight of composition A.

With increasing amount of paste-forming component(s), the viscosity of the composition can be adjusted, in particular lowered. Good paste properties can be obtained, if the paste-forming component(s) are present in an amount of 10 to 60 wt. % or 10 to 50 wt. %.

Paste-forming components which can be used include components which do not comprise a radically-curable moiety, or components which comprise a radically-curable moiety and mixtures of both.

According to one embodiment, the composition comprises paste-forming components not comprising a radically-curable moiety.

Examples of paste-forming components not comprising a radically-curable moiety include components containing the structural units of glycols (including ethylene glycol, propylene glycol, butylene glycol), glycerine and alkyl ethers, block-co-polymers of ethylene glycol and propylene glycol (commercially available e.g., as Synperonic™ and Pluronic™, copolymers of ethylene glycol, propylene glycol and/or tetrahydrofuran, alkoxylated glycerine or alkoxylated pentaerythritol or other multifunctional alcohols, and mixtures thereof.

In particular, the following paste-forming component were found to be useful: polyethylene glycol, polypropylene glycol and mixtures thereof, in particular those having a molecular weight (Mw) in the range of 200 to 10,000 g/mol.

If present, paste-forming component(s) not comprising a radically-curable moiety are typically present in the following amounts: at least: 1 or 5 or 10 wt. %; utmost: 60 or 55 or 50 wt. %; range: 1 to 60 or 5 to 55 or 10 to 50 wt. %; wt. % with respect to weight of composition A.

According to one embodiment, the composition comprises paste-forming components comprising a radically-curable moiety.

Paste-forming components comprising a radically-curable moiety can be characterized by the following features alone or in combination: a) comprising at least one (meth)acrylate moiety; b) comprising a (poly)oxy alkylene moiety or backbone; c) being hydrophilic.

A combination of feature a) and b) is sometimes preferred.

Using components which are hydrophilic can be advantageous to facilitate an adequate moisture uptake of the composition during use.

Acrylate or methacrylate-functionalized monomers are suitable as basic components for free radical polymerization. Monomers can be mono-, di- or higher acrylate or methacrylate functionalized compounds and mixtures of monomers can be used.

Suitable examples of the paste-forming components comprising a radically-curable moiety include liquid (meth)acrylate functionalized homo- or copolymers of ethylene glycol, propylene glycol and/or THF and mixtures thereof.

Hydrophilic monomers which can also be used include acid functionalized monomers such as mono- or dimethacrylate-functionalized tartaric acid or citric acid. Such components may positively contribute to the haemostatic properties of the composition.

More specific examples can be characterized by the following general formula with n being in a range of 3 to 15.

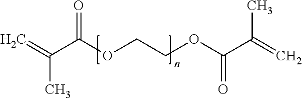

In particular, the following radically-curable components were found to be useful: polyethylene glycol dimethacrylate (Mₙ=330 g/mol), polyethylene glycol dimethacrylate (Mₙ=550 g/mol), polyethylene glycol dimethacrylate (Mₙ=750 g/mol), tetraethylene glycol dimethacrylate (Mₙ=302 g/mol), di-methacrylate functionalized copolymer of ethylene oxide and THF (Mₙ about 6,000 g/mol).

If present, the paste-forming components comprising a radically-curable moiety is present in the following amounts: at least: 3 or 5 or 10 wt. %; utmost: 75 or 70 or 68 wt. %; range: 1 to 60 or 5 to 55 or 10 to 50 wt. %; wt. % with respect to the weight of composition A.

Composition A may comprise an initiator or initiator system suitable for curing radically-curable components in addition.

An initiator or initiator system is typically present, if the composition comprises paste-forming components with a radically-curable moiety.

Radically-curable components are typically cured by way of a free-radical polymerization, with the free radicals being provided via suitable initiator systems photochemically, and/or by redox reactions.

According to one embodiment, the composition comprises a photo-initiator or photo-initiator system. The nature and structure of the photo-initiator system is not particularly limited unless the intended purpose is not negatively affected.

The photo-initiator is capable of generating free radicals for polymerization upon exposure to light. The photo-initiator(s) has typically a light absorption band in a wavelength range of 390 to 500 nm. Using a photo-initiator being soluble in the dental retraction composition described in the present text is preferred.

Photo-initiators which can be used typically contain a moiety selected form acyl phosphine oxids, benzoin ether, acetophenone, benzoyl oxime, phenylglyoxate, «-hydroxyketones or «-aminoketones.

A particularly suitable class of photo-initiators include the class of acylphosphine oxides, as described e.g. in U.S. Pat. No. 4,737,593 (Elrich et al.).

Such acylphosphine oxides can typically be characterized by the following formula

(R⁹)₂—P(═O)—C(═O)—R¹⁰

wherein each R⁹ individually can be a hydrocarbyl group such as alkyl, cycloalkyl, aryl, and aralkyl, any of which can be substituted with a halo-, alkyl- or alkoxy-group, or the two R⁹ groups can be joined to form a ring along with the phosphorous atom, and wherein R¹⁰ is a hydrocarbyl group, an S—, O—, or N-containing five- or six-membered heterocyclic group, or a —Z—C(═O)—P(═O)—(R⁹)₂ group, wherein Z represents a divalent hydrocarbyl group such as alkylene or phenylene having 2 to 6 carbon atoms.

Preferred acylphosphine oxides are those in which the R⁹ and R¹⁰ groups are phenyl or lower alkyl- or lower alkoxy-substituted phenyl. By “lower alkyl” and “lower alkoxy” is meant such groups having from 1 to 4 carbon atoms.

Suitable bisacylphosphine oxides can also be described by the following formula

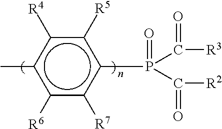

wherein n is 1 or 2, and R⁴, R⁵, R⁶ and R⁷ are H, C₁-4 alkyl, C₁-4 alkoxyl, F, Cl or Br; R² and R³, which are the same or different, stand for a cyclohexyl, cyclopentyl, phenyl, naphthyl, or biphenylyl radical, a cyclopentyl, cyclohexyl, phenyl, naphthyl, or biphenylyl radical substituted by F, Cl, Br, I, C₁-4 alkyl and/or C₁-4 alkoxyl, or an S or N-containing 5-membered or 6-membered heterocyclic ring; or R² and R³ are joined to form a ring containing from 4 to 10 carbon atoms and being optionally substituted by 1 to 6 C₁-4 alkyl radicals.

Examples include bis-(2,6-dichlorobenzoyl)phenylphosphine oxide, bis-(2,6-dichlorobenzoyl)-2,5-dimethylphenylphosphine oxide, bis-(2,6-dichlorobenzoyl)-4-ethoxyphenyl-phosphine oxide, bis-(2,6-dichlorobenzoyl)-4-biphenylylphosphine oxide, bis-(2,6-dichloro-benzoyl)-4-propylphenylphosphine oxide, bis-(2,6-dichlorobenzoyl)-2-naphthylphosphine oxide, bis-(2,6-dichlorobenzoyl)-1-napthylphosphine oxide, bis-(2,6-dichlorobenzoyl)-4-chlorophenyl-phosphine oxide, bis-(2,6-dichlorobenzoyl)-2,4-dimethoxyphenylphosphine oxide, bis-(2,6-dichlorobenzoyl) decylphosphine oxide, bis-(2,6-dichlorobenzoyl)-4-octylphenylphosphine oxide, bis-(2,6-dimethoxybenzoyl)-2,5-dimethylphenylphosphine oxide, bis-(2,6-dimethoxy-benzoyl)phenylphosphine oxide, bis-(2,4,6-trimethylbenzoyl)-2,5-dimethylphenylphosphine oxide, bis-(2,6-dichloro-3,4,5-trimethoxybenzoyl)-2,5-dimethylphenylphosphine oxide, bis-(2,6-dichloro-bis-(2-methyl-1-naphthoyl)-2,5-3,4,5-trimethoxybenzoyl)-4-ethoxyphenylphosphine oxide, dimethylphenylphosphine oxide, bis-(2-methyl-1-naphthoyl)phenylphosphine oxide, bis-(2-methyl-1-naphthoyl)-4-biphenylylphosphine oxide, bis-(2-methyl-1-naphthoyl)-4-ethoxyphenylphosphine oxide, bis-(2-methyl-1-naphthoyl)-2-naphthylphosphine oxide, bis-(2-methyl-1-naphthoyl)-4-propylphenylphosphine oxide, bis-(2-methyl-1-naphthoyl)-2,5-dimethylphosphine oxide, bis-(2-methoxy-1-naphthoyl)-4-ethoxyphenylphosphine oxide, bis-(2-methoxy-1-naphthoyl)-4-biphenylylphosphine oxide, bis-(2-methoxy-1-naphthoyl)-2-naphthylphosphine oxide and bis-(2-chloro-1-naphthoyl)-2,5-dimethylphenylphosphine oxide.

A preferred acylphosphine oxide is bis(2,4,6-trimethylbenzoyl)phenyl phosphine oxide (OMNIRAD™ 819, IGM Resin B. V., Waalwijk, Netherlands).

Suitable are also liquid blends of acylphosphine oxides with at least one other photo-initiator (available e.g. as OMNIRADIM 1000, OMNIRAD™ 2022, OMNIRAD™ 2100 or OMNIRAD™ 4265, IGM Resin B. V., Waalwijk, Netherlands).

If present, the initiator, in particular the photo-initiator system is present in the following amounts: lower amount: at least 0.01 or at least 0.05 or at least 0.1 wt. %; upper amount: at most 8 or at most 5 or at most 4 wt. %; range: 0.01 to 8 or 0.01 to 5 wt. % or 0.01 to 4 wt. %; wt. % with respect to the weight of composition A.

If present, the photo-initiator is typically activated with light having a wavelength in the visible range (e.g. 430 to 480 nm). A light device which can be used is e.g. 3M's Elipar™ DeepCure-S LED Curing light (3M Oral Care).

Composition A may also comprise a dye.

As a dye absorbs radiation, it is a means for helping to adjust the curing behaviour and in particular the curing depth of a radiation-curable composition, e.g. to ensure that only the upper section or layer of the composition is cured, but that the lower section or layer of the composition remains uncured or only in a partially cured stage.

Dyes which were found to be suitable may be characterized by the following features alone or in combination:
a) solubility: at least 0.1 g in 100 g triethylenglycole di(meth)acrylate (TEGDMA) at 23° C.;
b) having a light absorption maximum in the range of 420 to 600 nm;
c) having a fluorescence band in the range of 500 to 650 nm;
d) comprising a perylene or naphtalimide moiety.

A combination of the features a) and b) or a), b) and c) or a), b), c) and d) can sometimes be preferred.

Suitable examples include red dyes like Lumogen™ F Red 300 (BASF) and Fluoreszenzrot 94720 (Kremer) having an absorption maximum at about 575 nm, orange dyes like Fluoreszenzorange 94738 (Kremer) having an absorption maximum at about 526 nm and yellow dyes like Fluoreszenzgelb 94700 (Kremer) having an absorption maximum at about 474 or 476 nm or Lumugen™ F yellow 083 (BASF).

The dye may be present in the following amounts: at least: 0 or 0.005 or 0.01 wt. %; utmost: 1 or 0.8 or 0.5 wt. %; range: 0 to 1 or 0.005 to 0.8 or 0.1 to 0.5 wt. %; wt. % with respect to the weight of composition A.

Composition A described in the present text can also contain one or more additives.

Additives, which can be present in the composition include colourant(s), rheological modifier(s), surfactant(s), flavouring agent(s), antioxidant(s) or stabilizer(s), preserving agent(s), muco-adhesive components, mixtures and combinations thereof.

There is no need for additives to be present, however, if one or more additives are present, they are typically present in an amount which supports the intended purpose.

According to one embodiment, the composition has a colour which may allow an easy detection (especially in a patient's mouth compared to oral tissue and/or tooth substance) and control whether after the treatment all residues have been removed.

Examples of colourants which can be used include chinoline yellow dye (sicovit), chromophthalblue A3R, red iron oxide 3395, Bayferrox™ 920 Z Yellow, Neazopon™ Blue 807 (copper phthalocyanine-based dye), Helio™ Fast Yellow ER, Brilliant Blue FCF, Fast Green FCF and/or Orange Yellow S. Pigments or dyes which are stable under acidic conditions are preferred.

According to a further embodiment, rheology modifiers can be added. Rheology modifiers might contribute to the viscosity and effect the rinsability. Rheology modifiers which can be added include silicone oil.

In another embodiment, the composition may comprise one or more surfactants. Typical surfactants, which can be used, include anionic, cationic, zwitterionic or non-ionic surfactants.

There is no need for a surfactant to be present at all. However, if a surfactant is present, it is typically present in an amount of up to 2 wt. % or up to 1 wt. % or up to 0.05 wt. %, with respect to the weight of composition A.

In another embodiment, the composition may comprise a flavouring agent(s) to improve the taste and/or smell of the composition.

Typical flavouring agent(s), which can be used, include but are not limited to isoamylacetate (banana), benzaldehyde (bitter almond). Cinnamic aldehyde (Cinnamon), ethylpropionate (fruity), methyl anthranilate (Grape), mints (e.g. peppermints), limonene (e.g. Orange), allylhexanoate (pineapple), ethylmaltol (candy), ethylvanillin (Vanilla), methylsalicylate (Wintergreen).

There is no need for a flavouring agent to be present at all. However, if a flavouring agent is present, it is typically present in an amount of up to 3 wt. % or up to 0.1 wt. % of up to 0.01 wt. %, with respect to the weight of composition A.

The composition may also comprise an antioxidant or stabilizer(s).

Stabilizer(s) which can be used often comprise a phenol moiety or phosphonic acid moieties.

Specific examples of stabilizer(s) which can be used include: p-methoxyphenol (MOP), hydroquinone monomethylether (MEHQ), 2,6-di-tert-butyl-4-methyl-phenol (BHT; Ionol), pheno-thiazine, 2,2,6,6-tetramethyl-piperidine-1-oxyl radical (TEMPO) Vitamin E; N,N′-di-2-butyl-1,4-phenylenediamine; N,N′-di-2-butyl-1,4-phenylenediamine; 2,6-di-tert-butyl-4-methylphenol; 2,4-dimethyl-6-tert-butylphenol; 2,4-dimethyl-6-tert-butylphenol and 2,6-di-tert-butyl-4-methylphenol; 2,6-di-tert-butylphenol; pentaerythritoltetrakis(3-(3,5-di-tert-butyl-4-hydroxyphenyl) propionate) (previously known as Irganox™ 1010); octyl-3,5-di-tert-butyl-4-hydroxy-hydrocinnamate; octadecyl 3-(3,5-di-tert-butyl-4-hydroxyphenyl) propionate; 1,3,5-trimethyl-2,4,6-tris(3,5-di-tert-butyl-4-hydroxybenzyl)benzene; 2,2′,4,4′-tetrakis-tert-butyl-3,3′-dihydroxybiphenyl; 4,4-Butylidenebis(6-tert-butyl-m-cresol); 4,4′-Isopropyliden-bis-(2-tert-butylphenol); 2,2′-methylene-1,3,5-tris(3,5-di-tert-butyl-4-hydroxybenzyl-)-1,3,5-triazine-1-bis(6-nonyl-p-cresol); 2,4,6 (1H,3H,5H)trione; pyrogallol; N-nitroso-N-phenylhydroxylamine; 3-propenylphenol, phenothiazine, N-phenyl-2-naphthylamine, phosphorous acid phenylphosphonic acid; vinylphosphonic acid or combinations or mixtures thereof.

To enhance the adhesion of composition A to the soft tissue in the moist oral environment muco-adhesive components could also be added such as cyanoacrylates, starch-based compositions or hydroxy cellulose.

Additive(s) are typically present in the following amounts: at least: 0 or 0.01 or 0.1 wt. %; utmost: 20 or 15 or 10 wt. %; range: 0 to 20 or 0.01 to 15 or 0.1 to 10 wt. %; wt. % with respect to the weight of composition A.

Besides the components mentioned above, composition A may in addition comprise a component Y being able to react with agent X being contained in composition B wherein
component Y is either present in composition A before composition A is applied to the selected surface area; or
component Y is introduced into composition A after composition A has been applied to the selected surface area.

Reacting means that by bringing component Y and agent X together, a chemical reaction starts.

Reactions which may take place include production of gas, such as oxygen, nitrogen or carbon dioxide, or initiation of polymerization.

Examples for component Y include peroxide decomposition components, metal ion component comprising from ions of lithium, magnesium or calcium, initiator compounds.

Component Y can be present in the following amounts: at least: 0 or 0.01 or 0.1 wt. %; utmost: 10 or 5 or 1 wt. %; range: 0 to 10 or 0.01 to 5 or 0.1 to 1 wt. %; wt. % with respect to the weight of composition A.

In one embodiment, component Y comprises one or more hydrogen peroxide decomposition components.

The hydrogen peroxide decomposition component is a component which is able to decompose hydrogen peroxide or a hydrogen peroxide precursor thereby producing or generating oxygen.

Suitable hydrogen peroxide decomposition components include enzymes, Pt components, manganese dioxide, complexes of Fe(III), metalloporphyrin, mononuclear metal non-porphyrin, a dinuclear metal non-porphyrin, a trinuclear metal non-porphyrin, tetranuclear metal non-porphyrin and mixtures thereof.

In some embodiments, the component Y can include a peroxidase. In some embodiments, the component Y can include a catalase. Catalases can be found in a wide variety of eukaryotic and prokaryotic organisms including, but not limited to Agrobacterium tumefaciens, Aliivibrio salmonicida, Anopheles gambiae, Aspergillus nidulans, and Aspergillus niger. Catalases can catalyze the disproportionation of two molecules of hydrogen peroxide into two molecules water and one molecule oxygen.

Suitable hydrogen peroxide decomposition components are also described in US 2017/0367941 A1 (Haeberlein et al.), US 2020/0315942 A1 (Gong et al.), WO 2018/075149 A1 (3M). The content of these references is herewith incorporated by reference.

The hydrogen peroxide decomposition component may be present in the following amounts: at least: 0 or 0.01 or 0.1 wt. %; utmost: 5 or 3 or 1 wt. %; range: 0 to 5 or 0.01 to 3 or 0.1 to 1 wt. %; wt. % with respect the weight of composition A.

In certain embodiments, component Y includes a metal ion selected from the group of lithium ion (Li⁺), magnesium ion (Mg²⁺), calcium ion (Ca²⁺), and a combination thereof; and an aprotic base having a pK_{b} in water of greater than 15.4, or a precursor thereto. The aprotic base often is a halogen-containing anion. In certain embodiments, the aprotic base is a halide (e.g., iodide, bromide, and chloride).

In certain embodiments, the aprotic base of component A includes triflate ion (OTf), iodide ion (I), perchlorate ion (ClO₄⁻), bromide ion (Br⁻), chloride ion (Cl), or a combination thereof.

In certain embodiments, a precursor of a metal ion of component Y includes a solid salt (e.g., a lithium halide, a magnesium halide, a calcium halide, or a combination thereof). In certain embodiments, a precursor of an aprotic base having a pK_{b} in water of greater than 15.4 includes a solid salt (e.g., a lithium halide, a magnesium halide, a calcium halide, a sodium halide, a sodium triflate, a sodium perchlorate, or a combination thereof).

In certain embodiments, the metal ion and aprotic base of component Y are provided by the same compound. For example, in certain embodiments, component Y includes a lithium halide, a magnesium halide, a calcium halide, or a combination thereof. In certain embodiments, component Y includes LiCl, MgCl₂, CaCl₂), or a combination thereof.

The metal ion component may be present in the following amounts: at least: 0 or 0.01 or 0.1 wt. %; utmost: 5 or 3 or 1 wt. %; range: 0 to 5 or 0.01 to 3 or 0.1 to 1 wt. %; wt. % with respect the weight of composition A.

Composition A may also be characterized by the following properties alone or in combination:
a) viscosity: 2 to 100 at 23° C. and measured with a shear rate of 50 s-1;
b pH value: 4 to 9 if brought in contact with water,
c) water uptake: at least 150% with respect to the weight of composition A.
   If desired, the respective features can be determined as described in the example section. A combination of the following properties is sometimes preferred: a) and b); a) and c); b) and c).

Composition A described in the present text may comprise, consist essentially of or consists of the following components:
guanidinyl-containing polymer: 1 to 60 wt. %,
carrageenan: 1 to 40 wt. %,
filler: 5 to 70 wt. %,
paste-forming component: 1 to 60 wt. %,
additives: 0 to 20 wt. %,
wt. % with respect to the weight of composition A.

In a further embodiment composition A may comprise, consist essentially of or consists of the following components:
guanidinyl-containing polymer in an amount of 1 to 60 wt. %,
carrageenan in an amount of 1 to 40 wt. %,
filler in an amount of 5 to 70 wt. %,
paste-forming component in an amount of 1 to 60 wt. %,
radically curable components being different from the paste-forming component in an amount of 0 to 60 wt. %,
a photo-initiator system in an amount of 0 to 8 wt. %,
component Y in an amount of 0 to 5 wt. %,
dyes in an amount of 0 to 1 wt. %,
additive(s) in an amount of 0 to 10 wt. %,
wt. % with respect to the weight of the composition.

According to another embodiment composition A may comprise, consist essentially of or consists of the following components:
guanidinyl-containing polymer in an amount 5 to 40 wt. %,
carrageenan in an amount of 8 to 35 wt. %,
filler in an amount of 10 to 60 wt. %,
paste-forming component in an amount of 10 to 50 wt. %,
radically curable components being different from the paste-forming component in an amount of 10 to 50 wt. %,
a photo-initiator system in an amount of 0.1 to 8 wt. %,
a component Y in an amount of 0.01 to 1 wt. %.
dyes in an amount of 0.01 to 1 wt. %,
additive(s) in an amount of 1 to 8 wt. %,
wt. % with respect to the weight of the composition.

During the application process an active substance referred to as agent X is brought into contact with composition A.

Agent X is present in composition B.

Besides agent X, composition B typically contains a solvent for dissolving agent X.

Liquids or solvents for dissolving agent X comprise, essentially consist of or consist of water. Thus, composition B is typically an aqueous composition.

As composition A has liquid absorbing properties, composition B comprising agent X is absorbed by composition A.

After application of composition A to a selected surface area agent X is dissolved out of the hydrogel formed by the guanidinyl-containing polymer and carrageenan and can provide a treatment effect.

Agent X is typically present in composition B in the following amounts: at least: 0.01 or 0.5 or 1 wt. %; utmost: 20 or 15 or 10 wt. %; range: 0.01 to 20, or 0.5 to 15, or 1 to 10 wt. %; wt. % with respect to the weight of composition B.

Agent X can be a chemically reactive component or comprise a medically effective component.

Agent X can be selected from a variety of components including hydro peroxide or hydro peroxide precursor, carbonate or bicarbonate component, antibiotics, vasoconstrictors, disinfection substances, anti-inflammatory substances, haemostatic substances, tooth conditioning substances, fluoride components, anti-tartar components, healing promotion components, antimycotic components, local anesthetic components, probiotic components, and mixtures thereof.

Agent X may be a hydrogen peroxide or hydrogen peroxide precursor.

In some embodiments, agent X includes hydrogen peroxide. The hydrogen peroxide can be generated by a peroxide generating enzyme in combination with the corresponding substrate, e.g., glucose oxidase and Superoxide Dismutase (SOD). For example, glucose oxidase can catalyze the oxidation of glucose to hydrogen peroxide. In some embodiments, the hydrogen peroxide may be in a form of a hydrogen peroxide adduct, such as carbamide peroxide, percarbonate salts or acids and poyvinylpyrrolidone (PVP) peroxide and combinations thereof. Suitable percarbonate salts or acids can include, but are not limited to percarbonic acid, sodium percarbonate, potassium percarbonate, magnesium percarbonate, calcium percarbonate, zinc percarbonate.

In some embodiments, agent X can include a hydrogen peroxide precursor, such as perborate salts or acids, metal peroxides, organic peroxide, inorganic peroxyacids or salts and combinations thereof. Suitable perborate salts or acids can include, but are not limited to perboric acid, sodium perborate, potassium perborate, magnesium perborate, calcium perborate, and zinc perborate. Suitable metal peroxides can include, but are not limited to, calcium peroxide and magnesium peroxide. Suitable organic peroxides can include, but are not limited to peroxycarboxylic acids, such as peracetic acid or salts thereof, permalonic acid or salts thereof, pertartaric acid or salts thereof and percitric acid or salts thereof. In some embodiments, the organic peroxide can be a peracetate salt or acid. Suitable inorganic peroxyacids or salts can include, but are not limited to peroxymonosulfuric acid, peroxyphosphoric acid and a potassium salt of a sulfuric peroxyacid.

In certain embodiments, component Y includes a carbonate or bicarbonate component or ions thereto.

In certain embodiments, a precursor of a bicarbonate ion of component Y includes a solid salt (e.g., potassium bicarbonate (KHCO₃) and sodium bicarbonate (NaHCO₃)). In certain embodiments, a precursor to a bicarbonate ion includes a carbonate and an acid, which can form the bicarbonate. For example, component Y can include a mixture of sodium carbonate plus hydrochloric acid, which forms sodium chloride and sodium bicarbonate.

Agent X may be an antibiotic agent.

Suitable antibiotic agents include doxycycline, penicillin, tetracycline, minocycline, amoxicilline, metronidazole, clindamycin, and combinations thereof. Examples of antibiotics can also include those described in U.S. Pat. No. 6,685,921 (Lawlor).

Antibiotic agents can be in particular useful for e.g. treating periodontitis.

Agent X may comprise a vasoconstrictor.

Suitable vasoconstrictors include tetrahydrozoline, epinephrine, norepinephrine, phenyl-ephrine, dopamine and mixtures thereof.

Agent X may comprise a disinfection substance.

Suitable disinfection, antiseptics, preservatives substances include chlorhexidine, hexetidine, triclosan, polyhexamethylene biguanide, octenidine, quaternary ammonium salts and polymers thereof, parabens, polyvidone iodine and combinations thereof.

Disinfection substances can be used e.g. for disinfecting the sulcus pockets and provide healing effects on periodontic conditions and gingiva inflammation.

Agent X may comprise an anti-inflammatory substance.

Suitable anti-inflammatory substances include ibuprofen, diclofenac, acetylsalicylic acid cortisones such as triamcinolone, prednisone or prednisolone, herbal substances (e.g. Yunnan Baiyao, Camilla extract, Sage extract, etc.) and mixtures thereof.

The use of anti-inflammatory substances may facilitate the healing process.

Agent X may comprise a anti mycotic component to treat conditions like oral candidiasis.

Suitable anti mycotic components include nystatine, amphotericine fluconazole, miconazole and mixtures thereof.

Agent X may comprise a haemostatic component.

Suitable haemostatic components include A1, Fe, Ca containing components, and mixtures thereof. A preferred haemostatic component is A1 sulphate.

E.g. components that deliver Ca²⁺ could be beneficial to haemostasis by promoting the natural hemostatic cascade.

Agent X may comprise a tooth conditioning substance.

Teeth conditioning substances which can be used include acids, dental primers or adhesives and mixtures thereof.

### Healing Promotion Substances

Agent X may comprise a healing promotion substance.

Suitable healing promotion substances include panthenol, hyaluronic acid, protein-free haemodialysate from calves' blood or herbal components (e.g. Myrrh or rhubarb).

Agent X may comprise a fluoride component.

The application of fluoride components to tooth surfaces may contribute to treating caries or helping to prevent getting caries.

Fluoride components which can be used include sodium fluoride, potassium fluoride, stannous fluoride, ammonium fluoride, silver diamine fluoride and mixtures thereof. Sodium fluoride and silver diamine fluoride are sometimes preferred.

Examples of fluoride salts can include those described in U.S. Pat. No. 6,685,921 (Lawlor), U.S. Pat. No. 3,535,421 (Briner et al.), and U.S. Pat. No. 3,678,154 (Briner et al.).

Agent X may comprise a local anaesthetic component.

The application of local anaesthetic components to oral surfaces may provide pain relieve for the patient in conjunction to other dental treatments e.g in case of periodontal treatment or teeth cleaning or bleaching procedures.

Suitable local anaesthetic component include lidocaine, benzocaine, ambroxole.

Agent X may comprise an anti-tartar component.

Examples of tartar control agents can include those described in U.S. Pat. No. 6,685,921 (Lawlor). Anti-tartar agents known for use in dental care products can include, but are not limited to, phosphate. Phosphates can include pyrophosphates, polyphosphates, polyphosphonates, and mixtures thereof. Pyrophosphate salts can include the dialkali metal pyrophosphate salts, tetra-alkali metal pyrophosphate salts, and mixtures thereof.

Agent X my comprise probiotic components.

Probiotic components may be useful for treating or prevent oral diseases such as gingivitis or periodontis.

Useful probiotic components include Lactobacillus reuteri Prodentis or Streptococcus salivarius K12.

The use of the composition described in the present text for the application of disinfectants (such as chlorhexidine, triclosan, polyhexamethylene biguanide, or polyvidone iodine), fluorides (in particular silver diamine fluoride), haemostatic components (in particular aluminium sulphate), or local anaesthetic components (such as lidocaine or benzocaine) to selected surface areas in the oral cavity of a mammal was found to be particular useful.

Such a composition is typically adjusted to a viscosity in the range of 20 to 100 Pa*s or 30 to 80 Pa*s determined at 23° C. and a shear rate of 50 1/s.

Composition A described in the present text does typically not contain water in an amount of 5 wt. % or more.

The compositions described in the present text can be produced by combining and/or mixing the respective components.

If radically-curable components and a photo-initiator are present, the mixing should be conducted under save light conditions.

The invention also relates to a kit of parts.

The kit of parts comprise or consists essentially of or consists of a Part A and a Part B and optionally an instruction for use. The instruction for use typically contains hints or instructions how the kit of parts should be used, which process steps should be followed and for what purpose the kit is intended for.

Part A comprises composition A as described in the present text.

Part B comprises composition B as described in the present text.

Parts A and B may further differ from each other by the size, shape and/or colour of the packaging materials used for storing the respective compositions.

During storage, the compositions (e.g. compositions A and B) described in the present text are typically packaged in a suitable packaging and delivery device.

The compositions are typically provided to the practitioner under hygienic conditions. One possibility to achieve this includes packing or storing the composition in a sealed container such as a capsule, cartridge or foil bag under hygienic conditions.

A suitable container typically has a front end and a rear end, a piston movable in the container and a nozzle or cannula for delivering or dispensing the composition located in the container. The container has usually only one compartment or reservoir.

A suitable container may have a volume in the range of 0.1 to 10 ml. This is the volume typically needed for a single application. Such a container is typically used only once (e.g. disposable packing).

If more than one application is desired, the container may have a larger volume, e.g. in the range of 1 ml to 50 ml.

The composition can be dispensed out of the container by moving the piston in the direction of the nozzle. The piston can be moved either manually or with the aid of an application device or applier designed to receive the container (e.g. an application device having the design of a caulk gun).

According to one embodiment, the composition of the present text is stored in a one-compartment delivery device.

Examples of containers which can be used include compules, syringes and screw tubes. Containers of this kind are exemplified in more detail e.g. in U.S. Pat. No. 5,927,562 (Hammen et al), U.S. Pat. No. 5,893,714 (Arnold et al.) or U.S. Pat. No. 5,865,803 (Major).

It can be advantageous, if a container is used comprising a nozzle having a shape and size, which allows an easy and safe application of the composition to the selected surface area of the tissue.

A device with a nozzle or cannula having an external diameter in the range of 0.6 mm to 1.3 mm and an internal diameter in the range of 0.2 mm to 0.9 mm has been found to be particular useful.

However, other shapes and diameters can be used as well, if the intended effect (i.e. widening of the sulcus) can be achieved.

It has been found that especially a container described in more detail in US 2011/151403 A (Pauser et al.) is useful for storing and dispensing the composition described in the present text.

Composition A is for use in a process of treating a mammal by applying an agent X to a selected surface area in the oral cavity of the mammal.

This process comprises the following steps:
a. applying composition A to a selected surface area in the oral cavity of the mammal,
b. bringing a composition B into contact with composition A, composition B comprising agent X,
c. optionally applying composition C to the surface of composition A, composition C comprising a further active agent,
d. letting composition A absorb at least a portion of composition B and optional composition C,
e. optionally curing composition A, if composition A contains radiation-curable components,
f. leaving composition A on the surface onto which composition A has been applied for at least 10 s,
g. removing composition A from the selected surface area.

The application of composition A to a selected surface area of the oral cavity is typically done with a device containing a nozzle.

Suitable devices include a syringe, a compule, or other packaging materials mentioned above.

Composition B (containing agent X) is brought in contact with composition A. Due to the liquid absorbing properties of composition A, composition B is at least partially absorbed by composition A.

This enables agent X to be in close contact with the surface area to be treated for a time sufficient for agent X to interact with the surface area.

The time period may be in a range of a few seconds to a few minutes, e.g. 10 s to 1h or 10 s to 30 min or 10 s to 1 min.

After the desired time period, composition A is removed from the selected surface area.

The removal can be performed by any suitable means, including wiping, brushing, rinsing, spraying, e.g. by using a water-air beam, which is typically included in a dental chair, or peeling in case the composition is in a cured state.

The respective compositions are typically applied in the following volumes: Composition A is applied in a volume of 0.1 to 2 ml per selected surface area; Composition B is applied in a volume of 0.1 to 2 ml per selected surface area; Composition C, is used, is applied in a volume of 0.1 to 2 ml per selected surface area.

If more than one selected surface area should be treated, the overall application volumes for compositions A and B increase.

By selected surface area is meant a specific surface area in particular in the oral cavity of a mammal. The size of such a selected surface area may range from 0.4 cm² to 4 cm² or from 0.8 cm² to 2 cm².

Compositions A and B can be applied in essentially the same volume or in different volumes.

According to one embodiment, composition A is applied in a huger volume compared to composition B. According to one embodiment, composition B is applied in a huger volume compared to composition A.

Examples of selected surface areas in the oral cavity include the sulcus of a tooth, the surface of a tooth (including dentin and/or enamel), the oral tissue surrounding a tooth, a tooth cavity, a root canal.

The composition described in the present text can or is to be used in a process of treating and/or preventing various diseases in the oral cavity of a mammal.

Diseases which can be treated or addressed include calculus, periodontitis, tissue lesions (including canker sores (aphthous ulcer), saliviary gland outlets (e.g. ductus glandula submandibularis, ductus glandula parotis), endodontic disorders, gingivitis, oral candidiasis, caries, pain.

The paste like structure of composition A combined with its optional ability to be cured on the surface makes it possible to cover tissue lesions more effectively. With that, moisture control can be provided in critical aeras.

The composition can be applied onto the area to be treated and, if desired, the composition can be cured on the outer surface to provide a secure sealing environment of the area.

Produced saliva from the salivary gland would be absorbed by the paste and the hydrogel is formed. The cured outer layer of the sealing protects it against liquids from the surrounding and against unintended mechanical removal. An incorporation of active substances to limit or inhibit the production of saliva in this area could also be an option.

The compositions described in the present text are in particular suitable for treating calculus.

Such a process typically comprises the following steps:
a) applying a composition A1 containing a guanidinyl-containing polymer, carrageenan, a paste-forming component and filler to a tooth having calculus on its surface,
b) bringing a composition B1 comprising hydrogen peroxide or a hydrogen peroxide precursor into contact with composition A1,
c) bringing a composition C1 comprising a hydrogen peroxide decomposition component into contact with composition A1,
d) leaving composition A1 on the treated surface for at least 10 s, in particular for a time sufficient for the oxygen generated by the hydrogen peroxide decomposition component from the composition comprising hydrogen peroxide or a hydrogen peroxide precursor to migrate into the calculus,
e) removing composition A1 from the treated surface,
f) optionally removing the calculus from the tooth, f)
wherein steps b) and c) can also be carried out in reversed order, and
wherein the guanidinyl-containing polymer, carrageenan, the paste-forming component, the filler, the hydrogen peroxide or hydrogen peroxide precursor, and the hydrogen peroxide decomposition component are those as described in the present text.

According to one embodiment composition A1 for use in a process of treating calculus may comprise the following components: a hydrogen peroxide decomposition component, a guanidinyl-containing polymer, carrageenan, filler, a paste-forming component, optionally additives, wherein the respective components are such as described in the present text.

If composition A1 already comprises a hydrogen peroxide decomposition component, in the process described above, process step c) can be omitted.

The respective components of composition A1 are typically present in the following amounts:
a) hydrogen peroxide decomposition component: 0.01 to 20 wt. %,
b) guanidinyl-containing polymer: 1 to 60 wt. %,
c) carrageenan: 1 to 40 wt. %,
d) filler: 5 to 70 wt. %,
e) paste-forming component: 1 to 60 wt. %,
f) additives: 0 to 20 wt. %,
wt. % with respect to the weight of the composition.

According to one embodiment, composition A1 is characterized as follows:
a) the hydrogen peroxide decomposition component
   being selected from enzymes, Pt components, manganese dioxide, complexes of Fe(III), metalloporphyrin, mononuclear metal non-porphyrin, a dinuclear metal non-porphyrin, a trinuclear metal non-porphyrin, tetranuclear metal non-porphyrin or a mixtures thereof, and
   being present in an amount of 0.01 to 20 wt. %,
b) the guanidinyl-containing polymer
   the polymer of the guanidinyl-containing polymer being selected from polyvinylamine, poly(N-methylvinylamine), polyallylamine, polyallylmethylamine, polydiallylamine, poly(4-aminomethylstyrene), poly(4-aminostyrene), poly(acrylamide-co-methylamino-propylacrylamide), poly(acrylamide-co-aminoethylmethacrylate), polyethylenimine, polypropyleneimine, polylysine, polyaminoamides, polydimethylamine-epichlorohydrin-ethylenediamine, polyaminosiloxanes, dendrimers formed from polyamidoamine and polypropylenimine, biopolymers, polyacrylamide homo- or copolymers, amino-containing polyacrylate homo- or copolymers, and being present in an amount of 1 to 30 wt. %,
c) carrageenan
   being selected from iota-carrageenan, and
   being present in an amount of 1 to 30 wt. %,
d) filler
   being selected from silicates, quartz, cristobalite, alumina, magnesia, titania, and glasses, and
   being present in an amount of 0 to 60 wt. %,
e) the paste-forming component
   comprising components containing the structural units of glycols, glycerine, alkyl ethers, block-co-polymers of ethylene glycol and propylene glycol, copolymers of ethylene glycol, propylene glycol and/or tetrahydrofuran, alkoxylated glycerine or alkoxylated pentaerythritol or other multifunctional alcohols, and mixtures thereof, and being present in an amount of 15 to 60 wt. %,
f) additives being present in an amount of 0 to 15 wt. %, wt. % with respect to the weight of the composition.

Composition A described in the present text can also be used for the treatment of tissue lesions.

The sponge like structure of composition A is able to up moisture being present in the oral cavity and build a good healing environment.

If composition A contains radiation curable components and thus allows for a partial curing of the top layer of the composition, the hardened top layer contributes to protecting the inflamed tissue area and help to reduce pain for the patient.

The compositions and processes described in the present text are also suitable for endodontic treatments or the treatment of endodontic disorders.

After the inflamed or necrotic pulp tissue incl. nerve is removed and the root canal has been cleaned, medical treatments to soothe inflammation are typically applied. These treatments take time and the affected tooth is just temporary filled during this time.

The property of composition A described in the present text to function as a carrier that can be loaded with one or more (bio)active substances makes composition A useful in the medical treatment step of an endodontic procedure.

Furthermore, composition A with its swelling capacity might be especially beneficial for application into an endodontic canal with hard to reach areas.

By swelling, composition A can fill the root canal or tooth cavity completely and can adapt to the surrounding tissue.

Since composition A is just placed temporarily in the root canal, the easy rinsing property of composition A simplifies the removal and facilitates the clinical procedure as well.

The option to cure the top surface of composition A may further simplify the procedure as well since there might be no need to cover the open canal with a temporary filling or if a temporary filling is placed at the top, the solid film of composition A may allow a good separation from the filling material.

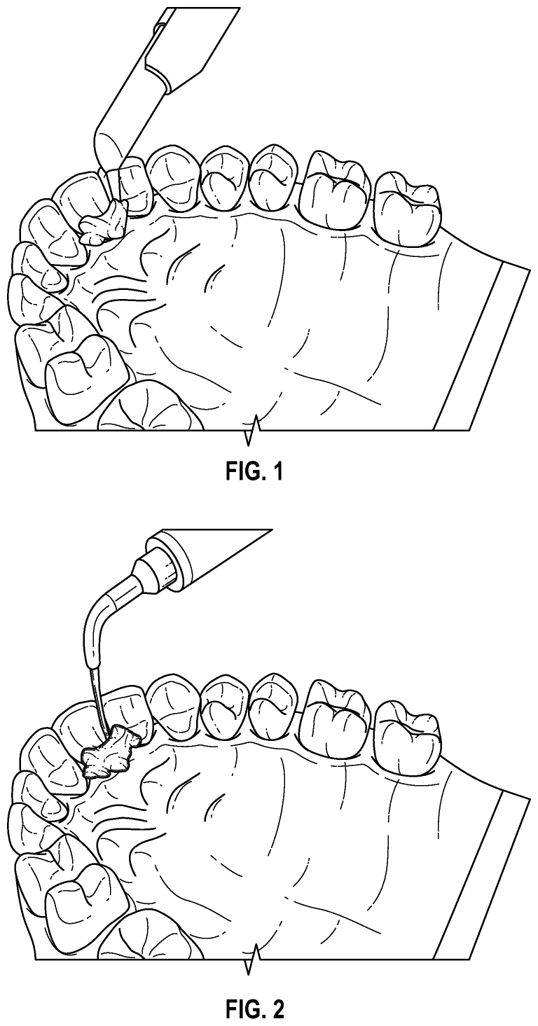

The process of application and use described in the present text is further exemplified by FIGS. 1 to 5.

In FIG. 1 the composition is applied from a syringe or compule with a small nozzle to a tooth having calculus on its surface. For better visibility the composition is coloured (e.g. blue colour). The applied composition has a volume of about 0.3 ml. The applied composition contains the component being able to release oxygen from a hydroperoxide containing solution.

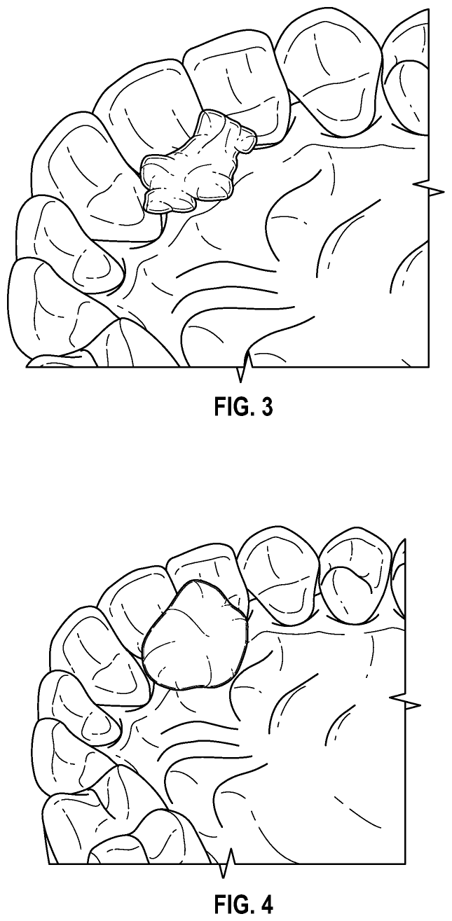

In FIG. 2 the hydroperoxide containing solution is applied on the tooth area having calculus on its surface. The application can be done either onto the composition, the area between the composition and the tooth, the sulcus or a combination of either of these areas. The hydroperoxide containing solution is essentially immediately absorbed by the composition. The composition thereby undergoes a volume expansion up to about 500%.

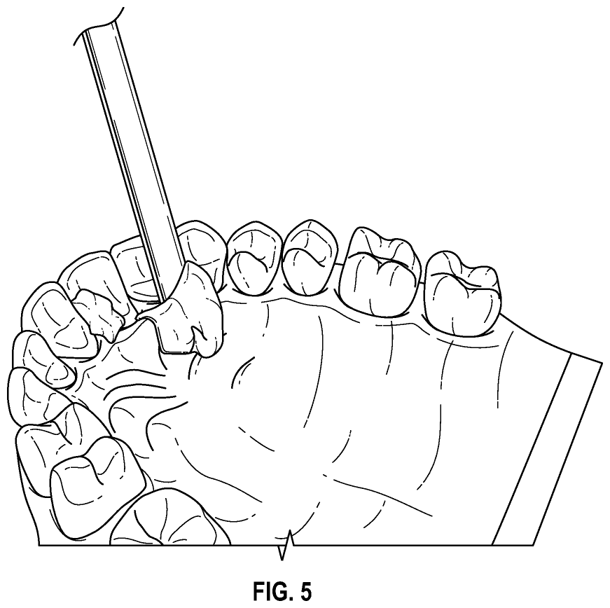

As shown in FIG. 4 the generation of oxygen starts. The composition is left in place for a time sufficient for enabling the generated oxygen to penetrate or migrate into the calculus.

After the treatment, the composition is removed from the tooth, e.g. by wiping, brushing, rinsing or spraying (FIG. 5).

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. The above specification, examples and data provide a description of the manufacture and use of the compositions and methods of the invention. The invention is not limited to the embodiments disclosed herein. One skilled in the art will appreciate that many alternative embodiments of the invention can be made without departing from the spirit and scope of thereof.

The following examples are given to illustrate the invention.

### Examples

Unless otherwise indicated, all parts and percentages are on a weight basis, all water is de-ionized water, and all molecular weights are weight average molecular weight. Moreover, unless otherwise indicated all experiments were conducted at ambient conditions (23° C.; 1013 mbar).

### Methods

### Viscosity

If desired, the viscosity can be measured using a Physica Rheometer MCR 302 device with a plate-plate system (diameter 20 mm) and a measuring gap width of 0.20 mm. The viscosity values (Pa*s) is typically recorded at 23° C. for each shear rate (starting from 10 1/s to 100 1/s in steps of 10 1/s). For comparing viscosity numbers, the viscosity in Pats at 50s-1 can be used.

### Particle Size (Suitable for Micro-Sized Particles)

If desired, the particle size distribution including the particle size (d50) per volume can be determined by laser diffraction with a Mastersizer 2000 (Malvern) particle size detection device applying the Fraunhofer approximation. During the measurement, ultrasonic is typically used to accurately disperse the sample. For water-insoluble particles, water is typically used as dispersant.

### pH Value

If desired, the pH value of can be determined as follows: 1.0 g of a component or composition is dispersed in 10 ml de-ionized water and stirred for about 5 min. A calibrated pH electrode is dipped into the suspension and the pH value is determined during stirring.

Alternatively, wet pH-sensitive paper can be used.

### Water Uptake

If desired, the water uptake properties can be determined as follows: 0.10g of the paste is placed in 0.50g water (app. 20° C.). The paste stays in contact with water for 2.0 min. Then the paste is removed and the weight of the swollen paste is measured on a scale. The water uptake is calculated as percentage increase of the final weight in relation to the original weight (0.10g).

### Shore Hardness

If desired, the Shore A hardness of the compositions can be determined according to DIN 53505:2000-08 and measured 10 min after start of light curing. All samples are light-cured for 20 sec from both sides using a Elipar™ DeepCure L with a LED emitting at 430-480 nm and 1480 mW/cm². The specimens were directly after the end of light-curing, put into an Otoflash device under Argon atmosphere for 1000 flashes.

### Catalase Activity

The component to be tested are brought into contact. If 2 liquid compositions (B+C) are to be used in combination with component (A) a waiting time of Imin in after the application of the first component is mandatory.

The enzymatic activity of the catalase in the test system is evaluated by a visual assessment on the intensity and timing of bubble development after the contact of a Hydrogenperoxide Solution in water following an evaluation scheme.

| Rating | Meaning |
|---|---|
| ++ | Immediate and very intense bubble or gas development |
| + | Immediate or slightly delayed (<5 sec.) and/or slightly less |
| | intense bubble or gas development |
| − | Delayed (>5 sec.) and/or low intensity bubble or gas |
| | development |
| −− | No bubble or gas development visible |

### Materials

TABLE 1

| Component | | Description |
|---|---|---|
| Guanidinylated polyethyleneimine (g-PEI), | g-PEI | Guanidinyl-containing polymer |
| (preparation as described in the experimental part | | |
| of WO 2018/128704 A1) | | |
| Irganox ™ 1010 | IRGA | Antioxidant (sterically hindered phenol) |
| Gelcarin ™ PH 379 | i-CAR | Iota-Carrageenan |
| Polyethylene glycol (Mw = 400 g/mol) | PEG400 | Paste forming liquid 1 (non-aqueous) |
| Mica; particle size: d70 = 1-3 μm | MICA | Filler; layer type 1:2 silicate mineral |
| Kaolin; particle size: d50 = about 14 μm | KAO | Filler, layer type 1:1 silicate mineral |
| Cristobalit powder | CRB | Sikron SF 6000 - HPF Minerals |
| Aerosil ™ R202 | Silica | Filler; pyrogenic silica |
| Omnirad ™ 2022 (IGM Resins) | | Photo-initiator |
| α-ω-methacrylated copolymer of | DIOL-MA | Radically-curable component; Paste forming |
| ethylenoxide/THF; Mw about 6,300 g/mol; | | liquid 2 (non-aqueous) |
| Diurethane dimethacrylate; 471 g/mol; | DUDMA | Radically-curable component; Paste forming |
| ethylenoxide/THF; Mw about 6,300 g/mol; | | liquid 3 (non-aqueous) |
| Pigment mixture (blue) - 10 wt. % pigment; 90 | Pigment | |
| wt. % mica | | |
| Catalase Powder - from bovine liver 2000-5000 | CAT | peroxide decomposition component, (Sigma |
| iU/mg | | Aldrich) |
| 25% Aluminium sulfat solution | Orbat | Orbat Sensitive (lege artis pharma GmbH + |
| | | Co KG - Dettenhausen/Germany) |
| Chlorhexidine digluconate solution 0.2% | CHX | Chorhexamed Praxis 0.2% (GSK) |
| Omnirad ™ 2022 (iGM Resins; Phosphine oxide | PO | Photo-initiator |
| Hydrogenperoxide solution | H2O2 | 3.0 wt % Hydrogenperoxide solution in water |

### General Process for Producing the Composition A

Paste-forming liquid and guanylated polyethylene imine were mixed. Then carrageenan was added and mixed. Then filler was added and mixed. All compositions were prepared in a vacuum speedmixer DAC-600.1 VAC at p<100 mbar.

### General Process for Producing the Composition C

The catalase was dissolved in water by stirring for 5 min. at room temperature.

The following compositions were produced:

Composition A

| | A1 | A2 | A3 | A4 |
|---|---|---|---|---|
| Component | wt. % | wt. % | wt. % | wt. % |
| PEG400 | 33.05 | 34.70 | 34.70 | 3.00 |
| DIOL-MA | | | | 50.07 |
| DUDMA | | | | 16.50 |
| IRGA | 0.10 | 0.10 | 0.10 | |
| g-PEI | 19.05 | 20.00 | 20.00 | 10.00 |
| i-CAR | 18.76 | 19.70 | 19.70 | 10.00 |
| Silica | 0.95 | 1.00 | 1.00 | 5.00 |
| KAO | 6.57 | 6.90 | — | |
| MICA | 16.50 | 17.32 | — | |
| CRB | — | — | 24.22 | |
| Pigment | 0.27 | 0.28 | 0.28 | |
| CAT | 4.76 | — | — | 5.00 |

Composition B

| | B1 | B2 | B3 |
|---|---|---|---|
| Component | wt. % | wt. % | wt. % |
| H₂O₂ | 100 | — | — |
| Orbat | — | 100 | — |
| CHX | — | — | 100 |

Composition C

| | C1 |
|---|---|
| Component | wt. % |
| water | 90 |
| CAT | 10 |

The compositions were further analysed with respect to water uptake capability, Shore hardness and catalase enzyme activity.

| | A1 | A2 | A3 | A4 |
|---|---|---|---|---|
| | wt. % | wt. % | wt. % | wt. % |
| Water uptake | 500 | 500 | 500 | 389 |
| | A1 | A2 | A3 | A4 |
| Shore hardness | n.a. | n.a. | n.a. | 92 |
| | Ex. 1 | Ex 2 | Ex. 3 | CE 1 |
| Component | A1 + B1 | A4 + B1 | A2 + B1 + C1 | A2 + B1 |
| Catalase Activity | ++ | ++ | ++ | −− |

CE: Comparative Example

The experimental compositions provided good liquid absorption properties with all aqueous solutions. After liquid uptake the pastes have swollen and were easy to remove from the treated surface area either with a spatula or a dental air-water spray.

1. A composition A for use in a process of treating a disorder in the oral cavity of a mammal,
composition A comprising a guanidinyl-containing polymer, carrageenan, filler, a paste-forming component, and optionally additives,
the process comprising the steps of
applying composition A to a selected surface area in the oral cavity of the mammal,
bringing a composition B into contact with composition A, composition B comprising agent X, letting composition A absorb at least a portion of composition B,
leaving composition A on the surface onto which composition A has been applied for at least 10 s, removing composition A from the selected surface area, agent X in composition B being selected from hydro peroxide or hydro peroxide precursor, carbonate or bicarbonate component, antibiotics, vasoconstrictors, disinfection substances, anti-inflammatory substances, hemostatic substances, tooth conditioning substances, fluoride components, anti-tartar components, healing promotion components, antimycotic components, local anesthetic components, probiotic components, and mixtures thereof.

2. (canceled)

3. The composition A for use according to claim 1, the polymer of the guanidinyl-containing polymer being selected from polyvinylamine, poly(N-methyl-vinylamine), polyallylamine, polyallylmethylamine, polydiallylamine, poly(4-aminomethylstyrene), poly(4-aminostyrene), poly(acrylamide-co-methylaminopropylacrylamide), poly(acrylamide-co-aminoethylmeth-acrylate), polyethylenimine, polypropyleneimine, polylysine, polyaminoamides, polydimethylamine-epichlorohydrin-ethylenediamine, polyaminosiloxanes, dendrimers formed from polyamidoamine and polypropylenimine, biopolymers, polyacrylamide homo- or copolymers, amino-containing polyacrylate homo- or copolymers.

4. The composition A for use according to the paste-forming component comprising components containing the structural units of glycols, glycerine, alkyl ethers, block-co-polymers of ethylene glycol and propylene glycol, copolymers of ethylene glycol, propylene glycol and/or tetrahydrofuran, alkoxylated glycerine or alkoxylated pentaerythritol or other multifunctional alcohols, and mixtures thereof.

5. The composition A for use according to claim 1, the optional additives being selected from colourant(s), rheological modifier(s), surfactant(s), flavouring agent(s), antioxidant(s) or stabilizer(s), preserving agent(s).

6. The composition for use according to claim 1, not comprising water in an amount of 5 wt. % or more with respect to the weight of the composition.

7. The composition A for use according to claim 1, composition A comprising the respective components in the following amounts:
guanidinyl-containing polymer: 1 to 60 wt. %,
carrageenan: 1 to 40 wt. %,
filler: 5 to 70 wt. %,
paste-forming component: 1 to 60 wt. %,
additives: 0 to 20 wt. %,
wt. % with respect to the weight of composition A.

8. The composition for use according to claim 1, composition A comprising in addition a component Y being able to react with agent X contained in composition B, wherein
component Y is either present in composition A before composition A is applied to the selected surface area; or
component Y is introduced into composition A after composition A has been applied to the selected surface area.

9. The composition for use according to claim 1, component Y being selected from a peroxide decomposition component, a metal ion component comprising ions of lithium, magnesium or calcium, acidic components, or initiator components.

10. The composition for use according to claim 1,
agent X in composition B comprising
a hydrogen peroxide or hydrogen peroxide precursor, or
carbonate or bicarbonate component,
component Y in composition A comprising
a peroxide decomposition component, or
a metal ion component comprising from ions of lithium, magnesium or calcium.

11. The composition for use according to claim 1, the paste-forming component comprising a radically-curable moiety, and
composition A comprising in addition optionally a photo-initiator.

12. The composition for use according to characterized by the following features alone or in combination:
a) composition A being applied in a volume of 0.1 to 2 ml per selected surface area;
b) composition B being applied in a volume of 0.1 to 2 ml per selected surface area;
c) composition A being left for a time period of 10 s to 1 h;
d) the removing of composition A being done by wiping, brushing, rinsing, spraying or peeling.

13. The composition A for use according to claim 1, the disorder of the mammal being selected from calculus, periodontitis, tissue lesions, endodontic disorders, gingivitis, oral candidiasis, pain, caries or combinations thereof.

14. A kit of parts for use in a process of treating a disorder in the oral cavity of a mammal, the kit of parts comprising Part A and Part B and optionally an instruction for use,
Part A comprising composition A,
Part B comprising composition B,
composition A and composition B being as described in any of the preceding claims.

15. A process of treating calculus in the oral cavity of a mammal, the process comprising the steps of
a) applying a composition A1 containing a guanidinyl-containing polymer, carrageenan, a paste-forming component and filler to a tooth having calculus on its surface,
b) bringing a composition B1 comprising hydrogen peroxide or a hydrogen peroxide precursor into contact with composition A1,
c) bringing a composition C1 comprising a hydrogen peroxide decomposition component into contact with composition A1,
d) leaving composition A1 on the treated surface for at least 10 s, in particular for a time sufficient for the oxygen generated by the hydrogen peroxide decomposition component from the composition comprising hydrogen peroxide or a hydrogen peroxide precursor to migrate into the calculus,
e) removing composition A1 from the treated surface,
f) optionally removing the calculus from the tooth,
wherein steps b) and c) can also be carried out in reversed order, and
wherein the guanidinyl-containing polymer, carrageenan, the paste-forming component, the filler, the hydrogen peroxide or hydrogen peroxide precursor, and the hydrogen peroxide decomposition component are those as described in any of the preceding claims.

16. The composition A for use according to claim 1 being characterized as follows:
a) agent X being a hydrogen peroxide decomposition component being selected from enzymes, Pt components, manganese dioxide, complexes of Fe(III), metalloporphyrin, 20 mononuclear metal non-porphyrin, a dinuclear metal non-porphyrin, a trinuclear metal non-porphyrin, tetranuclear metal non-porphyrin or a mixtures thereof, and being present in an amount of 0.01 to 20 wt. %,
b) the polymer of the guanidinyl-containing polymer being selected from polyvinylamine, poly(N-methylvinylamine), polyallylamine, polyallylmethylamine, polydiallylamine, 25 poly(4-aminomethylstyrene), poly(4-aminostyrene), poly(acrylamide-comethylamino-propylacrylamide), poly(acrylamide-co-aminoethylmethacrylate), polyethylenimine, polypropyleneimine, polylysine, polyaminoamides, polydimethylamine-epichlorohydrin-ethylenediamine, polyaminosiloxanes, dendrimers formed from polyamidoamine and polypropylenimine, biopolymers, polyacrylamide homo- or 30 copolymers, amino-containing polyacrylate homo- or copolymers, and being present in an amount of 1 to 30 wt. %,
c) carrageenan being selected from iota-carrageenan, and being present in an amount of 1 to 30 wt. %,
d) filler being selected from silicates, quartz, cristobalite, alumina, magnesia, titania, and 35 glasses, and being present in an amount of 0 to 60 wt. %,
e) the paste-forming component comprising components containing the structural units of glycols, glycerine, alkyl ethers, block-co-polymers of ethylene glycol and propylene glycol, copolymers of ethylene glycol, propylene glycol and/or tetrahydrofuran, alkoxylated glycerine or alkoxylated pentaerythritol or other multifunctional alcohols, and mixtures thereof, and being present in an amount of 15 to 60 wt. %,
f) additives being present in an amount of 0 to 15 wt. %, 5 wt. % with respect to the weight of the composition.
